(19) United States
(12) Patent Application Publication
(10) Pub. No.: US 2003/0055331 A1
(43) Pub. Date: Mar. 20, 2003

(54) Methods of endobronchial diagnosis using imaging

(75) Inventors: Robert Kotmel, Burlingame, CA (US); Peter Soltesz, San Jose, CA (US); Anthony Wondka, Mountain View, CA (US); Rodney Perkins, Woodside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PULMONx, Palo Alto, CA (US)

(21) Appl. No.: 10/241,974

(22) Filed: Sep. 11, 2002

Related U.S. Application Data
(60) Provisional application No. 60/322,366, filed on Sep. 11, 2001.

(51) Int. Cl. A61B 5/05

(52) U.S. Cl. 600/410; 600/443

(57) ABSTRACT

Devices and methods are provided for acquiring and analyzing an image data file to generate diagnostic information reflecting an individual lung compartment. A lung compartment could be an entire lobe, a segment or a subsegment and beyond, hereinafter subsegments and beyond will be referred to simply as segments. Such analysis is used to assess the level of disease of individual lung compartments, both for quantification of the disease state and for determining the most appropriate treatment plan. This analysis allows the imaging technology to be used as a functional diagnostic tool as well as an anatomical diagnostic tool. To this end, dynamic data or images may also be acquired at specific points throughout the breathing cycle. Since air movement in and out of a lung compartment during the breathing cycle is a direct indicator of lung function in some diseases like emphysema, analysis of images during the breathing cycle will indicate levels of disease. Thus, a physician may be able to determine the nature of the disease, severity of the disease and the most effective course of treatment from a computerized image of the lung.

### CROSS-REFERENCES TO RELATED APPLICATIONS

This application claims the benefit and priority of U.S. Provisional Patent Application No. 60/322,366 (Attorney Docket 017534-001900US), filed Sep. 11, 2001, the full disclosure of which is hereby incorporated by reference for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

1. Field of the Invention

The present invention relates generally to medical methods, systems, and kits. Particularly, the present invention relates to methods and apparatus for performing diagnostic testing on individual compartments of a lung. More particularly, the present invention provides for such testing with imaging technologies.

Chronic obstructive pulmonary disease (COPD) is a significant medical problem affecting 16 million people or about 6% of the U.S. population. Specific diseases in this group include chronic bronchitis, asthmatic bronchitis, and emphysema. In general, two types of diagnostic tests are performed on a patient to determine the extent and severity of COPD: 1) imaging tests and 2) functional tests. Imaging tests provide a good indicator of the location, homogeneity and progression of the diseased tissue. Images may be obtained by any standard imaging technique, such as computed tomography (CT), magnetic resonance imagining (MRI), polarized gas MRI, ultrasound, ultrasound with perfluroban, x-ray, or positive emission tomography (PET), to name a few. These imaging techniques generate a three-dimensional image of a body part, such as the lung, comprised of computerized data which can be stored, analyzed, manipulated and transmitted for a variety of uses. For example, during CT imaging, a CT scanner provides an x-ray source which rotates around the patient and each rotation produces a single cross-sectional image of a slice of the body. Incremental advancement of the patient allows a series of cross-sectional images to be taken which, when combined, create the three-dimensional image the body and the body part of interest. With some CT scanners, a scan of the lungs can be achieved in approximately 22 seconds with thin slices each in the range of 2.5 to 5.0 mm.

However, these traditional imaging tests do not give a direct indication of how the disease is affecting the patient's overall lung function and respiration capabilities. This can be measured with functional testing, such as spirometry, plethesmography, oxygen saturation, and oxygen consumption stress testing, to name a few. Traditionally, these diagnostic tests are used together to determine the course of treatment for the patient.

Treatment may include a variety of options, one such option is Lung Volume Reduction (LVR) which typically involves resecting diseased portions of the lung. Resection of diseased portions of the lungs both promotes expansion of the non-diseased regions of the lung and decreases the portion of inhaled air which goes into the lungs but is unable to transfer oxygen to the blood. Minimally invasive techniques may be used to isolate target lung tissue segments from other regions of the lung. In this instance, isolation is achieved by introducing an access catheter endotracheally or thorascopically to the target air passage of the lung. The target lung tissue segment is then collapsed by aspirating air (and any other gases or liquids that may have been introduced) from the segment and optionally sealed off. Exemplary methods and systems to perform such isolation procedures are described U.S. patent application Ser. No. 09/606320 (Attorney Docket No. 017534-000710), incorporated herein by reference. See also U.S. Pat. No. 6,258,100.

Currently, the diagnostic tests are limited in the amount and type of information that may be generated. For example, diagnostic imaging may provide information to the physician regarding which lung segments “appear” more diseased, but in fact a segment that appears more diseased may actually function better than one that appears less diseased. Functional testing is performed on the lungs as a whole. Thus, the information provided to the physician is generalized to the whole lung and does not provide information about functionality of individual lung segments. Thus, physicians may find difficulty targeting interventional treatments to the segments most in need and to avoid unnecessarily treating segments that are not in need of treatment or less in need. In general, the diseased segments cannot be differentiated, prioritized for treatment or assessed after treatment for level of response to therapy.

For these reasons, it would be desirable to provide devices, methods and techniques which would overcome at least some of the shortcomings discussed above. In particular, it would be desirable to provide methods for utilizing conventional imaging files, such as CT scans, to diagnose, assess and monitor individual lung segments. Further, it would be desirable to generate information from the image data files related to functional assessment of the lung segments, to compare the collected and generated measurement information to diagnose the level of disease of the lung segments, determine the most appropriate treatment options and monitor the disease levels over time. In addition, it would be desirable to synchronize image generation or scanning with the breathing cycle of the patient. At least some of these objectives will be met by the inventions described hereinafter.

2. Description of the Background Art

Patents and applications relating to lung access, diagnosis, and/or treatment include U.S. Pat. Nos. 6,258,100, 6,174,323, 6,083,255, 5,972,026, 5,752,921; 5,707,352; 5,682,880; 5,660,175; 5,653,231; 5,645,519; 5,642,730; 5,598,840; 5,499,625; 5,477,851; 5,361,753; 5,331,947; 5,309,903; 5,285,778; 5,146,916; 5,143,062; 5,056,529; 4,976,710; 4,955,375; 4,961,738; 4,958,932; 4,949,716; 4,896,941; 4,862,874; 4,850,371; 4,846,153; 4,819,664; 4,784,133; 4,742,819; 4,716,896; 4,567,882; 4,453,545; 4,468,216; 4,327,721; 4,327,720; 4,041,936; 3,913,568 3,866,599; 3,776,222; 3,677,262; 3,669,098; 3,498,286; 3,322,126; EP 1078601, WO 01/13908, WO 01/13839, WO 01/10314, WO 00/62699, WO 00/51510, WO 00/03642, WO 99/64109, WO 99/34741, WO 99/01076, WO 98/44854, WO 95/33506, and WO 92/10971.

WO 99/01076 describes devices and methods for reducing the size of lung tissue by applying heat energy to shrink collagen in the tissue. In one embodiment, air may be removed from a bleb in the lung to reduce its size. Air passages to the bleb may then be sealed, e.g., by heating, to fix the size of the bleb. WO 98/49191 describes a plug-like device for placement in a lung air passage to isolate a region of lung tissue, where air is not removed from the tissue prior to plugging. WO 98/48706 describes the use of surfactants in lung lavage for treating respiratory distress syndrome.

Lung volume reduction surgery is described in many publications, including Becker et al. (1998) Am. J. Respir. Crit. Care Med. 157:1593-1599; Criner et al. (1998) Am. J. Respir. Crit. Care Med. 157:1578-1585; Kotloff et al. (1998) Chest 113:890-895; and Ojo et al. (1997) Chest 112:1494-1500.

The use of mucolytic agents for clearing lung obstructions is described in Sclafani (1999) AARC Times, January, 69-97. Use of a balloon-cuffed bronchofiberscope to reinflate a lung segment suffering from refractory atelectasis is described in Harada et al. (1983) Chest 84:725-728.

Improved apparatus, systems, methods, and kits for isolating lobar and sub-lobar regions of a patient's lungs is described in U.S. patent application Ser. No. 09/425272 (Attorney Docket No.: 017534-000600US). Once the lobar or sub-lobar region has been isolated, a variety of therapeutic and diagnostic procedures can be performed within the isolated region. Likewise, improved methods, systems, and kits for performing lung volume reduction in patients suffering from chronic obstructive pulmonary disease, or other conditions where isolation of a lung segment or reduction of lung volume is desired, is described in U.S. patent application Ser. No. 09/347032 (Attorney Docket No.: 017534-000700US), 09/606320 (Attorney Docket No.: 017534-000710US), and 09/898703 (Attorney Docket No.: 017534-000720US)

### BRIEF SUMMARY OF THE INVENTION

The present invention provides devices and methods for acquiring and analyzing lung images, usually image data files to generate diagnostic information reflecting an individual lung compartment. A lung compartment could be a lobe, a segment or a subsegment and beyond, hereinafter subsegments and beyond will be referred to simply as segments. Such analysis may be used to assess the level of disease of individual lung compartments, both for quantification of the disease state and for determining the most appropriate treatment plan. This analysis allows the imaging technology to be used as a functional diagnostic tool as well as an anatomical diagnostic tool. To this end, the invention also allows for dynamic data or images to be acquired at one or more specific points throughout a breathing cycle. Since air movement in and out of a lung compartment during the breathing cycle is a direct indicator of lung function in some diseases like emphysema, analysis of images during the breathing cycle will indicate levels of disease. Thus, a physician may be able to determine the nature of the disease, severity of the disease and the most effective course of treatment from a computerized image of the lung.

Methods of analyzing data in an image data file of a lung are provided by the present invention. As previously mentioned, when a lung is imaged by standard imaging techniques, such as computed tomography (CT), the resulting image is comprised of data which collectively is termed the image data file. According to the present invention, the image data file is analyzed using a controller, typically programmed with a software algorithm, which determines the periphery of at least one lung compartment within the lung based on the image data. This may be achieved by a variety of methods. In one embodiment, differences in density measurements throughout the lung are used to determine the borders or peripheries of the lung compartments. In another embodiment, differences in the sizes of lung passageways are used. Since passageways branch into passageways having smaller and smaller diameters, the passageways can be traced until the size of the passageways falls below a threshold value. At this point it is determined that the periphery of the lung compartment has been reached. In yet another embodiment, anatomical features are used to determine the periphery of a lung compartment. For example, a fissure between adjacent lobes may indicate the boundary of a lung compartment. In still another embodiment, the periphery of a lung compartment is determined based on the location of nearby lung compartments. Thus, as more and more compartments are identified, the peripheries of the remaining compartments can be identified based on extrapolation.

Once the periphery of a lung compartment is determined, the data representing the compartment may be isolated from the remainder of the image data.. The isolated compartment data may be used for a variety of purposes, such as presenting a visual image of the lung compartment independently of the remainder of the lung, calculating compartment volume, calculating density, assessing level of disease and comparing file data corresponding to different lung compartments.

Although a single image data file may be analyzed to provide functional information about a lung compartment, additional functional information may be derived by comparing and analyzing a series of image data files of a lung or lung compartment obtained throughout a patient's breathing cycle. Breathing patterns are commonly measured by spirometry, a test which is performed by breathing into an instrument known as a spirometer. The spirometer measures the volume and the rate of air that is inspired by a patient over a measured or specified time. The present invention utilizes data obtained from a spirometer to synchronize images captured within the patient's breathing cycle. For example, the imaging device may be activated to scan a patient's chest at specific points in the breathing cycle. Thus, a dynamic representation of the lung is provided while the lung is functioning. By comparing the images taken at these points, lung disorders that cause functional abnormalities can be identified.

Volume measurements by spirometry may also be used to calibrate software algorithms used in calculating lung compartment volumes based on image data files. The accuracy of the volume calculations made from the image data file data may be checked and corrected by comparison with the volume measurements obtained by the spirometer. To achieve this, the volume of the entire lungs is calculated from the image data file data. This value is compared to the volume measured for the lungs by the spirometer at the same point in the breathing cycle. A software algorithm is used to calibrate the calculations from the image data file data based on the difference in volume values.

Analysis of individual lung compartments may be further facilitated with the use of devices to directly access the lung compartments. For example, a radiopaque gas or liquid may be injected into the lung compartment to highlight the lung compartment during imaging. Such access may be achieved with the use of a pulmonary measurement system which uses a pulmonary catheter to directly access the lung compartment within the patient's anatomy. The pulmonary measurement system provides a variety of testing and imaging features which assist identification and assessment of lung compartments.

Measurements and/or calculated values may be presented in a data chart, typically displayed on a computer screen or any other visual display. Preferred embodiments of such a chart include various lung compartments and measurement values corresponding to each compartment. Thus, the lung compartments may easily be compared for severity of disease. Optionally, the lung compartments may be ranked in order of disease severity. This may serve as a guideline for treatment plans, such as minimally invasive treatments which isolate target lung tissue compartments from other regions of the lung. The most diseased compartments may be treated first or a combination of compartments with varying disease severity may be treated at once to provide the most effective treatment. To determine which compartment or combination of compartments may be most desired for treatment, a software algorithm which predicts the improvement in performance of the lung based on isolation of individual lung compartments may be used. Once determined, isolation can be achieved by introducing an access catheter endotracheally or thorascopically to the target air passage of the lung. The target lung tissue segment is then collapsed by aspirating air (and any other gases or liquids that may have been introduced) from the segment and optionally sealed off. The above described methods may be repeated after treatment to access the effectiveness of the treatment and to diagnose additional disease.

Other objects and advantages of the present invention will become apparent from the detailed description to follow, together with the accompanying drawings.

### BRIEF DESCRIPTION OF THE DRAWINGS

FIG. 1 is a schematic illustration of a three-dimensional image of a lung.

FIG. 2 depicts a lung compartment as a three-dimensional wire-framed image.

FIGS. 3A-3B depict examples of spirograms collected by a spirometer.

FIG. 4 illustrates a patient breathing into a spirometer which signals a CT scanner to create a scanned image of the patient's anatomy.

FIG. 5 is a perspective view of a pulmonary measurement system which may be used with the present invention.

FIG. 6 illustrates the use of a pulmonary catheter for accessing lung compartments.

FIG. 7 shows an example of a data chart for display of measurement values.

### DETAILED DESCRIPTION OF THE INVENTION

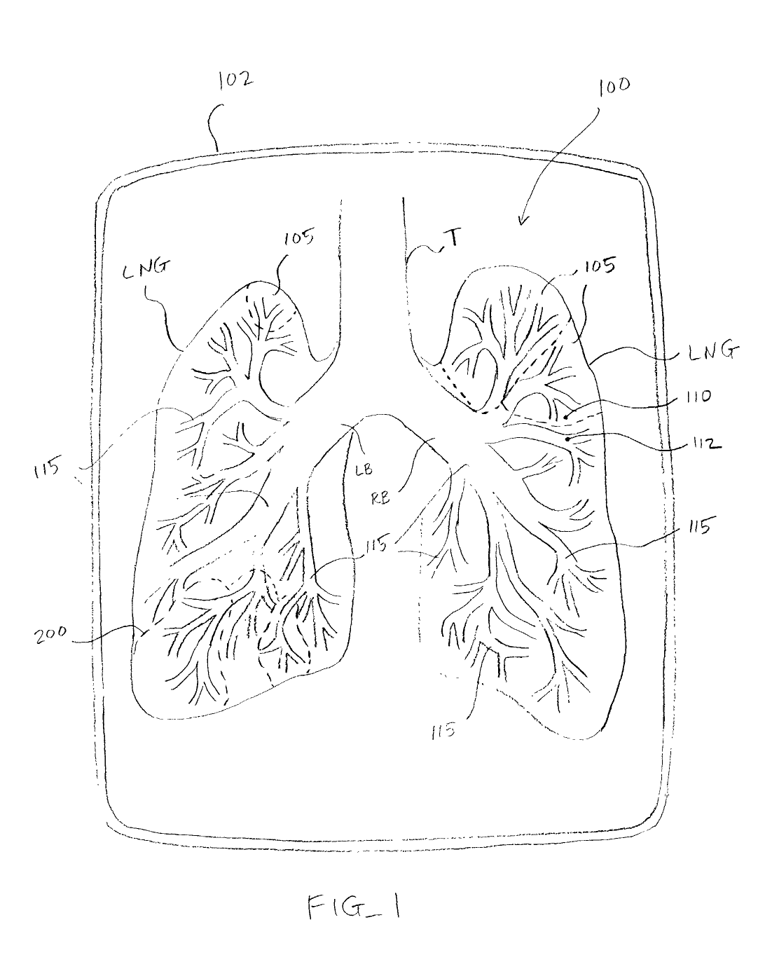

As stated previously, a variety of imaging techniques may be used to generate a three-dimensional image of a body part. FIG. 1 provides a schematic illustration of such an image 100, in this instance, of a lung LNG. The image 100 is the product of a image data file comprised of data which can be stored, analyzed, manipulated and transmitted for a variety of uses. One such use is to display the image 100 on a computer screen or visual display 102. The data can also be analyzed to identify individual lung compartments 105 within the lung LNG. Again, such compartments could be a lobe, a segment or a subsegment and beyond. Example compartments 105 are delineated by dashed lines in FIG. 1. By identifying individual compartments 105, each compartment can be isolated and analyzed to determine its level of disease and thus its contribution to the overall disease of the lungs.

To identify and isolate a compartment 105 of interest, a software algorithm determines the periphery of the lung compartment 105 within the lung LNG. This may be achieved by any suitable means or methods. In one embodiment, density measurements are used. The density of an area of tissue depicted in an image 100 can be calculated with the use of a software algorithm. Density can be determined by correlating the shade of the area of the tissue depicted in the image 100 with a density measurement based on known correlation standards. To determine the periphery of a lung compartment 105 using density measurements, a first location 110 and a second location 112 within the image 100 of the lung LNG are chosen. Typically, these locations 110, 112 are relatively close to one another as shown in FIG. 1 since it is estimated that a periphery exists between them. The density of the tissue is compared at the first location 110 with the density at the second location 110 to determine a difference in density. If the difference in density is above a density threshold value, is it determined that the locations 110, 112 are situated in different lung compartments, therefore defining at least a portion of a periphery of a lung compartment 105 between the locations 110, 112. If the difference in density is at or below the threshold value, it is likely that the locations 110, 112 are situated within the same lung compartment 105 and are not divided by a periphery of a lung compartment 105.

In another embodiment, the sizes of lung passageways 115 are used to determine the periphery of a lung compartment 105. As shown in FIG. 1, lung passageways 115 branch from the trachea T into the left bronchus and right bronchus LB and RB, respectively. The passageways 115 continue to branch throughout the lungs LNG, decreasing in size with each branch. If a lung compartment 105 is chosen to comprise a specific passageway and the branches descending therefrom, the periphery of the compartment 105 may be roughly identified as the region where the smallest diameter branches can be imaged, which may be approximately 1.0 mm. To define this periphery, a size threshold value is chosen to correspond with the size of passageways 115 in this region. Thus, lung passageways 115 are identified and their size determined as the passageways 115 branch. Size determinations may be achieved by direct measurement, extrapolation methods or other suitable means. Once the size falls below the size threshold value, at least a portion of the periphery of the lung compartment 105 is defined.

In yet another embodiment, an anatomical feature is used to determine the periphery of a lung compartment. An example of such an anatomical feature is a fissure between adjacent lobes. In this example, a lung compartment 105 may comprise a lobe wherein a fissure between the lobe and an adjacent lobe would anatomically signify an edge of the lobe and thus at least a portion of the periphery of the lung compartment. A software algorithm may be used to identify such an anatomical feature and define at least a portion of the periphery of the lung compartment based on the location of the anatomical feature.

And, in another embodiment, the periphery of a lung compartment is determined based on the location of the peripheries of nearby lung compartments. Referring again to FIG. 1, a first periphery 200 of a first nearby lung compartment 202 and a second periphery 204 of a second nearby lung compartment 206 are shown. Assuming that the lung compartment of interest 210 comprises the area between the compartments 202, 206, the periphery of the lung compartment of interest is estimated based on the first and second peripheries 200, 204. In fact, a portion of the periphery may be comprised of the first and second peripheries 200, 204.

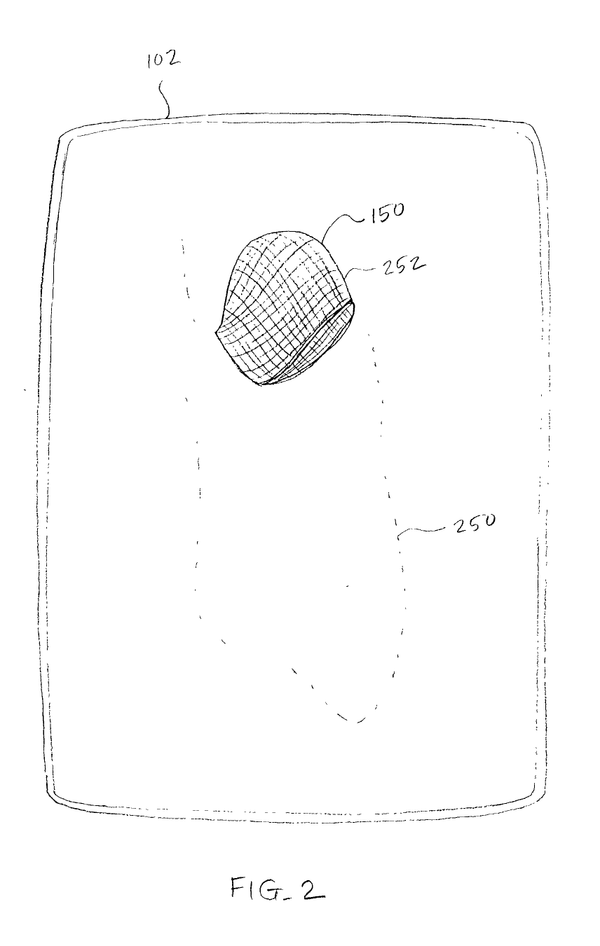

Once the periphery of the lung compartment 105 is determined and the compartment 105 of interest is defined, the compartment 105 may be isolated from the remainder of the lung LNG. Such isolation may be visual; to achieve this a software algorithm may be implemented which displays the image of the lung compartment 105 isolated from the lung. This is shown in FIG. 2 where the lung compartment 105 is depicted as a three-dimensional wire-framed image 252. The remainder of the lung LNG is depicted as a dashed line 250. Alternatively or in addition, such isolation may be physical wherein the image data corresponding the compartment 105 is copied, removed, separated or accessed independently of the remainder of the data. This isolated image data may be used for a variety of purposes, such as presenting a visual image, calculating compartment volume, calculating density, assessing level of disease and comparing image data corresponding to different lung compartments.

A variety of methods and techniques may be used to calculate the volume of a lung compartment 150. In one embodiment, voxels are defined within the lung compartment 150. A voxel is a volume measurement taken from an image calculated by multiplying the area of a small two-dimensional square on the image by the thickness of the tissue imaged, i.e. the thickness of the smallest slice of a CT scan. Typically, the dimensions of the two-dimensional square are equivalent to the thickness of the slice, for example the voxel dimensions would be 2 mm×2 mm×2 mm. Calculating the volume of a voxel can be achieved by known methods. By calculating the volume of each voxel and adding the volumes together, the volume of the lung compartment 105 is determined. This can be achieved with a software algorithm.

Likewise, a variety of methods and techniques may be used to calculate the density of a lung compartment 150. For example, as previously mentioned, the density of an area of tissue depicted in an image 100 can be calculated with the use of a software algorithm. Density can be determined by correlating the shade of the area of the tissue depicted in the image 100 with a density measurement based on known correlation standards. Density measurements can then be used to determine the level of disease in that area of tissue. Thus, each lung compartment 150 can be graded on level of disease, such as emphysema. Lung compartments 150 can then be ranked in order of disease severity for use in determining treatment options, such as determining the order in which to treat the lung compartments or determining which lung compartments should be treated for the most effective treatment protocol.

The image data file 100 used in the above described analyses may be obtained by a variety of imaging techniques, as previously mentioned. With many of these techniques, the image data file 100 is created while the patient is holding a breath to minimize movement and increase clarity of the image. Although such practice may allow some control over the point in which an image is taken during the breathing cycle, a more dynamic system of image capture is desirable for both accuracy and patient comfort. This may be achieved by synchronizing image capture with the patient's breathing pattern.

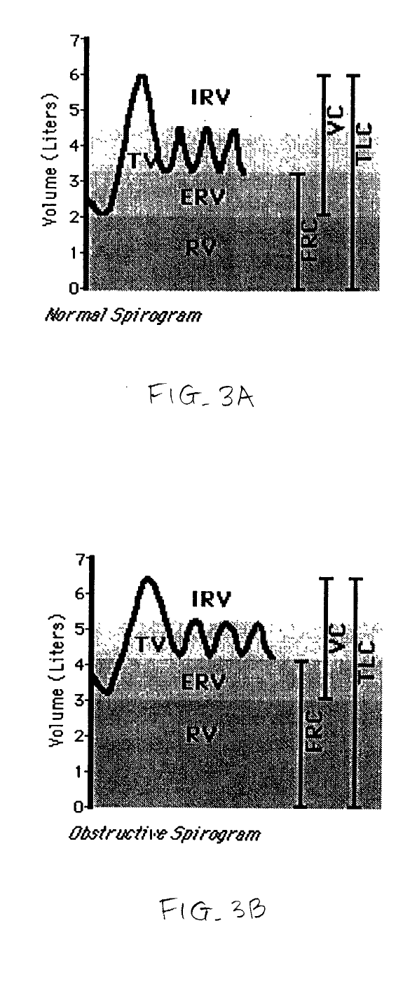

Breathing patterns are commonly measured by spirometry, a test which measures how well the lungs take in air, the volume of air the lungs hold, and how well the lungs exhale air. The information gathered during this test is useful in diagnosing certain types of lung disorders. The test is performed by breathing into an instrument called a spirometer that records the amount of air and the rate of air that is breathed in over a specified time. Some of the test measurements are obtained by normal breathing, and other tests require forced inhalation, such as Forced Inhaled Volume (FIV), and/or exhalation, such as Forced Exhaled Volume (FEV). FIGS. 3A-3B depict examples of spirograms or volumetric traces reflecting measurements collected by the spirometer. FIG. 3A illustrates a normal spirogram taken from a patient with no lung disorder. A variety of breathing volumes occurring during the breathing cycle are shown, such as Inspiratory Reserve Volume (IRV), Tidal Volume (TV), Expiratory Reserve Volume (ERV), Residual Volume (RV), Functional Residual Capacity (FRC), Vital Capacity (VC) and Total Lung Capacity (TLC). FIG. 3B illustrates an obstructive spirogram taken from a patient with an obstructive lung disorder such as emphysema. As shown, the spirogram is shifted upwards indicating, among others, a larger RV.

To synchronize image capture with the patient's breathing pattern, a spirometer is used to activate an imaging device to create an image data file at specific times in the breathing pattern. For example, the imaging device may be activated to scan a patient's chest at the point of peak inspiration during a patient's normal breathing cycle. Optionally, the imaging device may also be activated at other points, such as the end of inspiration, the end of exhalation, at maximum forced inspired volume, at maximum forced exhaled volume and during FEV over a standard length of time. By comparing scanned images taken throughout the breathing cycle, functional information may be derived. For example, lung disorders that cause functional abnormalities can be identified. In addition, the effects of obstructions, airway resistance, loss of elasticity, air trapping, inadvertent post end expiration pressure, and bronchopulmonary fistulas can be identified. Also, by comparing scanned images taken at the same point in the breathing cycle at different points in time, such as throughout the treatment protocol or post-treatment monitoring, improvement or worsening of disease may be determined.

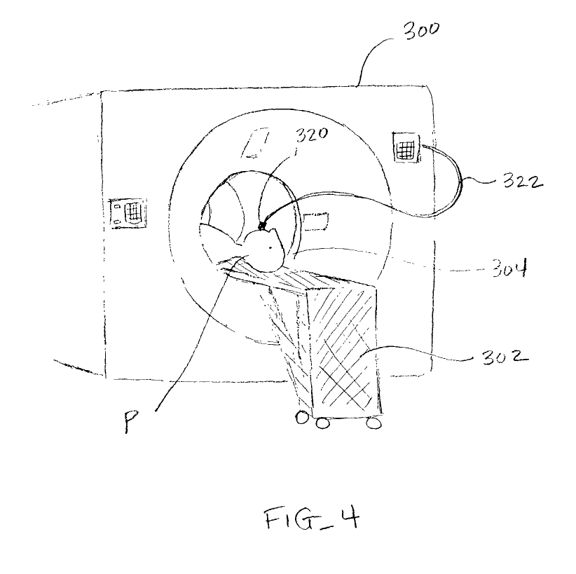

As described, spirometers generate pulmonary data upon receiving breath. To achieve synchronized image capture, a software algorithm is provided which generates at least one signal based on the pulmonary data. The imaging device is activated by the signal to create an image data file of the lung. In one embodiment, the imaging device is a CT scanner. As shown in FIG. 4, the CT scanner 304 is housed within a CT unit 300, which has a large circular hole in the center. The patient P is positioned on a scanning table 302 which is guided into the hole in the center of the CT unit 300. Typically, the CT scanner 304 rotates around the patient P and the patient P is repositioned longitudinally throughout the scan by moving the table 302 into or out of the hole. A spirometer 320 is placed in the patient's P mouth, as shown. As the patient breathes into the spirometer 320, pulmonary data, such as shown in FIGS. 3A-3B, is generated. A signal is generated at specific points in the breathing cycle to trigger the scanner 304 to scan the patient P at these points in time. To achieve this, a software algorithm generates the signal based on the pulmonary data from the spirometer. The scanner 304 is activated by the signal to create an image data file of the lung. The signal may be transmitted from the spirometer 320 to the scanner 304 by any suitable means. For example, the spirometer 320 may be connected to the CT unit 300 with the use of a cord 322 as shown. Or the spirometer 320 may be connected to a separate device, such as a computer, which is connected to the CT unit 300. Or, the spirometer 320 may be cordless and may transmit the signal with the use of infrared technology.

When the software algorithm generates a first signal at a first point in the breathing cycle so that a first image data file of the lung is created and a second signal at a second point in the breathing cycle so that a second image data file of the lung is created, a difference in lung volume may be quantified by comparing the first image data file with the second image data file. To assist in such quantification, a software algorithm can be used to calculate one or more of the breathing volumes previously described, such as IRV, TV, ERV, RV, FRC, VC, TLC, and FEV.

The images generated with these methods and the volumes calculated from the volumetric traces reflect the lungs as a whole. To analyze individual lung compartments, the previously described methods related to lung compartments are used. In addition, the calculations related to lung compartments may be calibrated with the use of the measurements related to the lungs as a whole. For example, the algorithm used to calculate the volume of a lung compartment can be used to calculate the volume of the total lungs. This calculation can be compared to the TLC value calculated based on the volumetric trace from the spirometer. Such comparison can calibrate the algorithm to ensure accurate calculations.

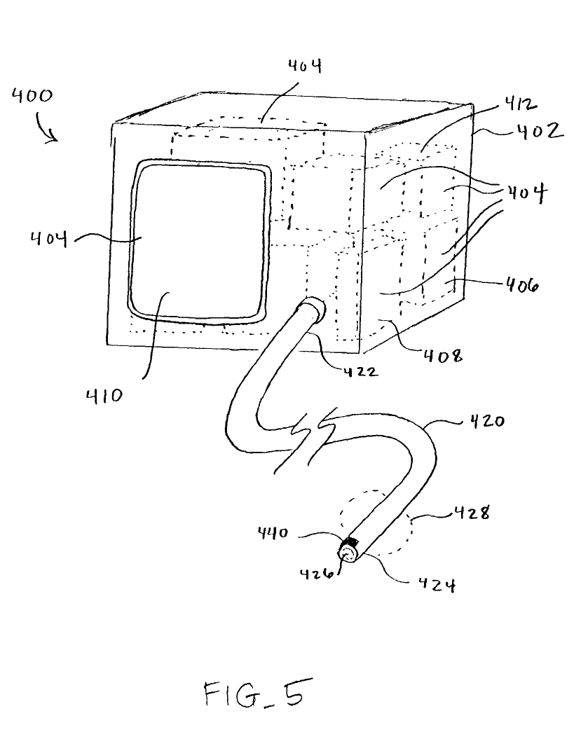

Analysis of individual lung compartments may be further facilitated with the use of devices to directly access the lung compartments. For example, a radiopaque gas or liquid may be injected into the lung compartment to highlight the lung compartment during imaging. This may be achieved with the use of a pulmonary measurement system comprising an Endobronchial Pulmonary Diagnostic (EPD) device and at least one measuring component connected with the device. An exemplary embodiment of such a pulmonary measurement system is described in copending U.S. patent application Ser. No. ______ (Attorney Docket No. 017534-001710US), incorporated by reference for all purposes. Referring to FIG. 5, the EPD device 402 comprises at least one measuring component 404, a number of which are shown in schematic form as dashed-lined boxes within the EPD device 402. Such measuring components 404 may take many forms and may perform a variety of functions. For example, the components 404 may include a gas dilution unit 406, an imaging unit 408, a visual display 410, an aspiration component 412, and mechanisms for measuring pulmonary mechanics or physiologic parameters, to name a few.

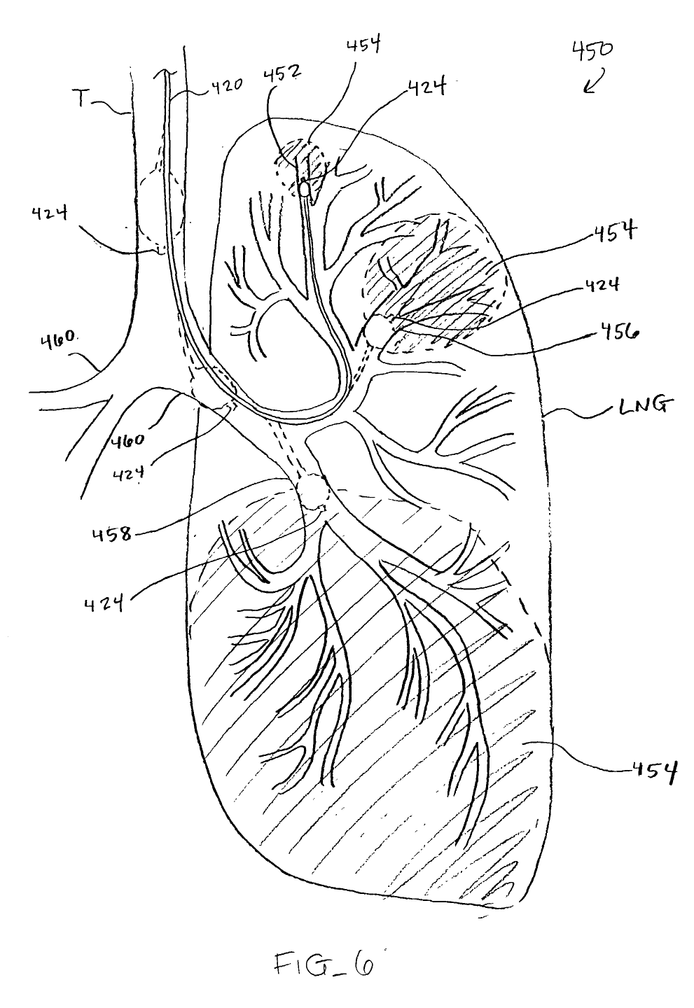

As shown, a pulmonary catheter 420 is removably attachable to the EPD device 402. Here, the catheter 420 is shown as having a proximal end 422, distal end 424, and an optional lumen 426 therethrough and occlusion member 428, both shown in dashed-line. As illustrated in FIG. 6, the catheter 420 is configured for introduction into the pulmonary anatomy 450, particularly into a bronchial passageway. As shown, the catheter 420 may be introduced into the bronchial passageways of a lung LNG to any depth. For example, as shown in solid line, the catheter 420 may be introduced so that it's distal end 424 is positioned within a distant lung segment 452 of the branching passageways. Inflation of the occlusion member 428 near its distal end 424 seals off the lung passageway around the catheter 420 leading to an individual lung compartment 454. In this position, the catheter 420 can isolate and measure a compartment 454 of the lung LNG, illustrated by a shaded dashed-lined circle. This provides direct communication with the lung compartment 454, isolated from the remainder of the lung.

In general, the components 404 include mechanical, electrical, chemical or other means to generate measurement data which characterizes the compartment of the lung which is being measured. For example, a component 404 may include a gas source and a pump which are used to fill the compartment with the gas for pressure or volume measurement. Typically, a component 404 works in conjunction with one or more sensors 440 which are located at any location within the measurement system. The component 404 may collect data from the sensor 440 and utilize the data in further measurement functions. Or, the component 404 may simply display the data on a visual display 410 or readout.

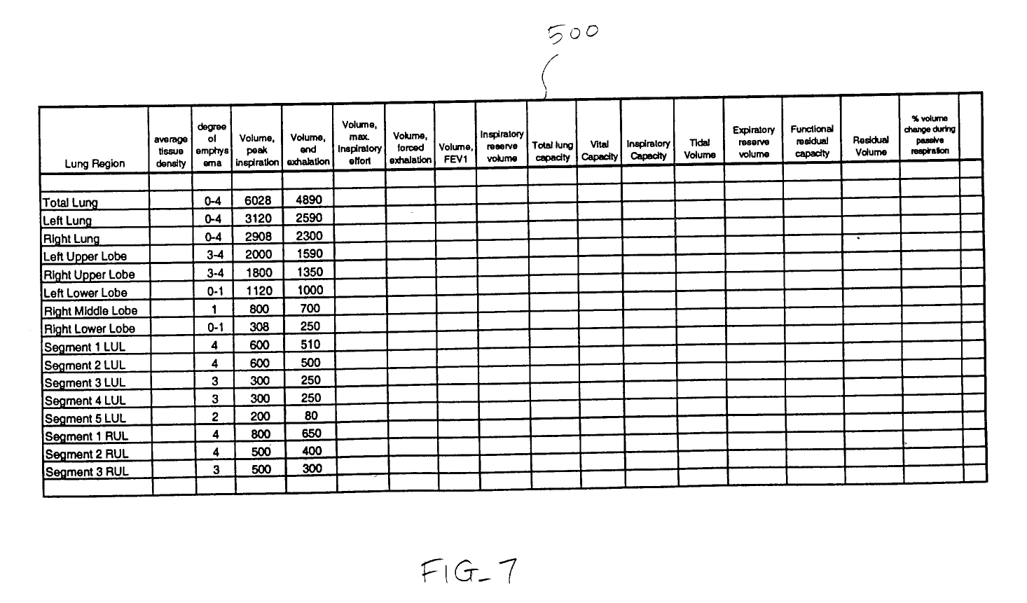

Measurements and/or calculated values collected and generated by any of the above described methods may be displayed on the visual display 102, the visual display 410 of the EPD device 402 or on any other visual display screen. Referring to FIG. 7, the values may be displayed in a data chart 500 as shown. Here, lung regions or compartments are identified and calculated or measured values are shown for each compartment. The values may be automatically displayed in the chart 500 and/ or values may be entered by the user. For example, a degree of emphysema rating, such as shown in the third column of the chart 500, may be entered by the user based on visual examination of images or examination of certain values in the chart 500. In addition, images, graphs, and other related information can also be displayed on the visual display screen. Thus, the lung compartments may be easily compared and ranked in order of disease severity. This may serve as a guideline for treatment plans, such as minimally invasive treatments which isolate target lung tissue compartments from other regions of the lung. For example, the most diseased compartments may be treated first or a combination of compartments with varying disease severity may be treated at once to provide the most effective treatment. To determine which compartment or combination of compartments may be most desired for treatment, a software algorithm which predicts the improvement in performance of the lung based on isolation of individual lung compartments may be used. Once determined, isolation can be achieved by introducing an access catheter endotracheally or thorascopically to the target air passage of the lung. The target lung tissue segment is then collapsed by aspirating air (and any other gases or liquids that may have been introduced) from the segment and optionally sealed off. The above described methods may be repeated after treatment to access the effectiveness of the treatment and to diagnose additional disease.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

What is claimed is:

1. A method of analyzing data in an image data file of a lung comprising:
providing the image data file of the lung to a computer; and
analyzing the image data file on the computer with an algorithm which determines the periphery of at least one lung compartment within the lung.

2. A method as in claim 1, further comprising analyzing the image data with the computer using an algorithm which calculates the volume of the lung compartment.

3. A method as in claim 2, wherein analyzing the image data comprises:
defining voxels within the periphery of the lung compartment;
calculating the volume of each voxel; and
adding the volumes of the voxels together.

4. A method as in claim 1, further comprising analyzing the image data with the computer using an algorithm which determines the density of tissue in the lung compartment.

5. A method as in claim 4, wherein the algorithm correlates the image shade with density of the tissue.

6. A method as in claim 4, further comprising grading the lung compartment for level of emphysema based on the density of the tissue in the compartment.

7. A method as in claim 1, further comprising analyzing the image data with the computer using an algorithm which displays an image of the lung compartment isolated from the lung.

8. A method as in claim 1, wherein analyzing the image data comprises:
determining the density of the tissue at a first location within the lung;
determining the density of the tissue at a second location within the lung; and
comparing the density at the first location with the density at the second location to determine a difference in density,
wherein at least a portion of the periphery is based on a difference in density between the first and second locations above a density threshold value.

9. A method as in claim 1, wherein analyzing image data comprises:
identifying a lung passageway within the lung; and
determining the size of the lung passageway,
wherein at least a portion of the periphery of the lung compartment is based on the size of the passageway.

10. A method as in claim 1, wherein analyzing the image data comprises:
identifying an anatomical feature on the image which signifies a natural division between lung compartments,
wherein at least a portion of the periphery of the lung compartment is based on the location of the anatomical feature.

11. A method as in claim 1, wherein analyzing image data comprises:
determining a first periphery of a first lung compartment; and
determining a second periphery of a second lung compartment,
whereas the periphery of the lung compartment is based on the first and second peripheries.

12. A method as in claim 1, wherein providing the image data file involves scanning the lung with the use of computer tomography, magnetic resonance imaging, ultrasound, x-ray or positive emission tomography.

13. A method of generating an image data file of a lung of a patient at at least one preselected point in a breathing cycle, said method comprising:
providing a spirometer which generates pulmonary data representing a breathing cycle;
providing a controller which generates a signal at at least one point in a breathing cycle based on the pulmonary data;
providing an imaging device which is activated by the signal to create an image of the lung; and
breathing into the spirometer so that the pulmonary data is generated and the at least one signal is generated to activate the imaging device to create the image of the lung.

14. A method as in claim 13, wherein the image comprises an image data file.

15. A method as in claim 13, wherein the pulmonary data comprises a volumetric trace of a breathing cycle.

16. A method as in claim 13, wherein the controller generates a first signal at a first point in the breathing cycle so that a first image of the lung is created and a second signal at a second point in the breathing cycle so that a second image of the lung is created.

17. A method as in claim 16, further comprising calculating a quantitative difference in lung volume by comparing the first image with the second image.

18. A method as in claim 15, further comprising calculating at least one breathing volume from the volumetric trace wherein the breathing volume is selected from the group consisting of total lung capacity, vital capacity, inspiratory reserve volume, tidal volume, inspiratory capacity, expiratory reserve volume, functional residual capacity and residual volume.

19. A method as in claim 14, further comprising analyzing data in the image data file to determine the periphery of at least one lung compartment within the lung.

20. A method as in claim 19, further comprising calculating the volume of the lung compartment based on the image data file.

21. A method as in claim 20, wherein the further comprising calculating the volume of the lung, comparing the calculated volume of the lung with the total lung capacity, and calibrating the controller.
